(19) United States
(12) Patent Application Publication
(10) Pub. No.: US 2006/0155309 A1
(43) Pub. Date: Jul. 13, 2006

(54) Sliding surgical clip

(75) Inventors: Amir Loshakove, Moshav-Bazra (IL); Ido Kilemnik, Herzelia (IL)

Correspondence Address: WOLF, BLOCK, SCHORR & SOLIS-COHEN LLP, 250 PARK AVENUE, NEW YORK, NY, 10177, US

(73) Assignee: By-Pass, Inc, Orangeburg, NY (US)

(21) Appl. No.: 10/529,125

(22) PCT Filed: Sep. 25, 2003
(86) PCT No.: PCT/IL03/00770

§ 371 Date: Mar. 24, 2005

(30) Foreign Application Priority Data
Sep. 25, 2002 (IL) IL02/00790

(51) Int. Cl. A61B 17/08

(52) U.S. Cl. 606/151

(57) ABSTRACT

A sliding surgical clip adapted to connect blood vessel tissue, comprising: an elongate rail leg having a main axis and terminating with a first tissue penetrating tip; and a sliding leg terminating with a second tip and configured to slide along said rail leg towards said first tissue penetrating tip, such that said two tips face each other and engage vascular tissue between them.

### RELATED APPLICATIONS

The present application claims priority from and is a continuation-in-part of PCT application PCT/IL02/00790, filed on Sep. 25, 2002, now published as WO 03/02647, and is also a continuation-in-part of PCT/IL01/00903, filed on Sep. 25, 2001. The disclosures of both these applications, which designate the US and were filed in English, are incorporated herein by reference.

### FIELD OF THE INVENTION

The present invention relates to surgical clips.

### BACKGROUND OF THE INVENTION

PCT publications WO 97/40754 and WO 97/28749, the disclosures of which are incorporated herein by reference, describe various staplers for coronary bypass surgery. In such clips, the clipping action is provided by distortion of the clips. However, such distortion may be difficult to apply in some situations, for example, where the clips may be undesirably distorted or where it is difficult to apply force in a desired controlled direction.

### SUMMARY OF THE INVENTION

An aspect of some embodiments of the invention relates to a two part surgical clip comprising two legs. A first leg slides along the other leg, which acts as a rail for guiding the first leg, when the clip changes from an open configuration to a closed configuration. In an exemplary embodiment of the invention, the sliding first leg is adapted to slide over the other leg and is locked in place by a locking mechanism. Optionally, the locking mechanism is a one-way lock which only prevent reverse sliding but not further sliding. In an exemplary embodiment of the invention, the sliding leg comprise a bent leg with an eye the encircles the other leg. In an exemplary embodiment of the invention, the two legs end in at least one sharp point, each said sharp points being adapted to pierce a blood vessel wall.

Optionally, the other leg is contiguous with or coupled to a tearing mechanism which tears the other leg near the lock, after the two legs are locked.

In an exemplary embodiment of the invention, an anastomosis clip delivery system is provided in which a plurality of clips are arranged to surround a graft. Optionally, the delivery system is adapted to retract the plurality of rail legs. The retracting and/or the tearing may be simultaneous for all rail legs. Alternatively, the retracting and/or the tearing may be serial for some or all of the rail legs.

Optionally, the tearing assists in locking the legs. In an exemplary embodiment of the invention, the tearing causes elongation of the torn section. Optionally, the elongated section is not exactly in the middle of a contiguous section of material (e.g., steel) or the tearing forces are not applied along the middle line, so the elongation is asymmetrical with respect to the middle and causes a bending of the tom end towards the middle line of the section. In some embodiments, locking is helped by bending towards the center of the leg. In other embodiments, the bending is away from a center of the leg.

In an exemplary embodiment of the invention, the rail leg includes one or more thickenings or widenings and one or more corresponding slots, so that when the eye of the sliding leg slides over a thickening, the thickening is pushed into the slot so that its overall width fits within the eye. Suitable design of the thickening, for example for the thickening to be gradual in the sliding direction and abrupt in the other direction, allows such a thickening to function as a lock. Optionally, a plurality of such thickenings are used to provide multiple locking positions.

In an alternative embodiment, the sliding leg slides within the rail leg, rather than outside of it.

In some embodiments of the invention, the sliding leg is adapted to not penetrate the blood vessel, for example by its being flat and not having a piercing point at its end. Optionally, one or both of the legs may be forked and/or may include a tissue stop to control a penetration depth thereof.

In an exemplary embodiment of the invention, the sliding leg is not a planar element that lies on a surface of a blood vessel. For example, the sliding leg may be bent. Optionally, the sliding leg is formed of a planar material and then bent out of plane. Optionally, a base section of the sliding leg lies parallel to a main plane of the rail leg. In an alternative embodiment, the base section is perpendicular to the main plane. In an alternative embodiment, the base section is curved.

An aspect of some embodiments of the invention relates to a set of clips for an anastomotic connection, in which individual clips includes eyes for a suture which interconnects the clips. In an exemplary embodiment of the invention, the eye is provided on a section of a clip which is positioned so that it may fall into the blood stream. Such a suture is optionally removed after the anastomosis connection is completed.

There is thus provided in accordance with an exemplary embodiment of the invention, a sliding surgical clip adapted to connect blood vessel tissue, comprising:

an elongate rail leg having a main axis and terminating with a first tissue penetrating tip; and

a sliding leg terminating with a second tip and configured to slide along said rail leg towards said first tissue penetrating tip, such that said two tips face each other and engage vascular tissue between them. Optionally, said second tip comprises a tissue penetrating tip. Alternatively or additionally, the clip comprises a tissue stop on said sliding leg, which stop prevents over penetration of said leg into vascular tissue.

Optionally, said second tip is forked.

In an exemplary embodiment of the invention, said second tip has a section between 0.5 mm and 5 mm long adapted to enter a blood vessel. Optionally, said second tip has a section between 1 mm and 4 mm long adapted to enter a blood vessel. Optionally, said second tip has a section between 0.5 mm and 3 mm long adapted to enter a blood vessel in an exemplary embodiment of the invention, said first tip has a section between 0.5 mm and 4 mm long adapted to enter a blood vessel.

In an exemplary embodiment of the invention, said first tip is forked.

In an exemplary embodiment of the invention, said first leg and said second leg are narrower than 1 mm, in a widest dimension thereof, for at least 3 mm adjacent their tips.

In an exemplary embodiment of the invention, said first leg and said second leg are narrower than 0.7 mm, in a widest dimension thereof, for at least 2 mm adjacent their tips.

In an exemplary embodiment of the invention, said first leg and said second leg are narrower than 1 mm, in a widest dimension thereof, for at least 20 mm adjacent their tips.

In an exemplary embodiment of the invention, said clip is adapted for use with a blood vessel having a diameter of between 2 mm and 40 mm.

In an exemplary embodiment of the invention, said clip is provided as a set of connectors arranged in a generally circular array and adapted for use for attaching a blood vessel having a diameter of between 1 mm and 10 mm to a second blood vessel. Optionally, said set is sutured together at their rail sections. Optionally, said second tip is adapted to penetrate vascular tissue without tearing the tissue.

In an exemplary embodiment of the invention, said elongate rail leg comprises a lock which prevents reverse sliding of said sliding leg. Optionally, said elongate rail leg comprises multiple lock locations which prevents reverse sliding of said sliding leg. Alternatively or additionally, said lock comprises a transaxial extension of said elongate rail which is configured to elastically move out of the way when said sliding leg sides towards said first tip. Optionally, said rail includes a slot adjacent said extension, to support said elastic motion. Alternatively or additionally, said transaxial extension is robust enough to withstand a force of at least 1 Kg on said extension.

In an exemplary embodiment of the invention, said elongate rail leg comprises a lock which prevents forward sliding of said sliding leg after being locked.

In an exemplary embodiment of the invention, the clip comprises a tissue stop on said rail leg to prevent over-penetration of said leg into vascular tissue.

In an exemplary embodiment of the invention, said rail leg defines a weakened location adapted to be selectively torn when sufficient force is applied to opposite sides of said weakened location.

In an exemplary embodiment of the invention, said rail leg defines a temporary locking location distanced from said tip, configured to hold said sliding leg prior to said sliding.

In an exemplary embodiment of the invention, said sliding leg engages said rail leg from its outside.

In an exemplary embodiment of the invention, said rail leg defines a slot along its length and wherein said sliding leg engages said rail leg from said slot. Optionally, said rail leg shares an elongate section with an adjacent rail leg, on an opposite side of a tearing location from said tip.

In an exemplary embodiment of the invention, said sliding leg includes a base section coupled to said rail leg. Optionally, said base section defines an aperture, which aperture fits around said rail leg. Optionally, said fit is snug.

In an exemplary embodiment of the invention, said base section lies in a plane, which plane lies parallel to said main axis.

In an exemplary embodiment of the invention, said base section lies in a plane, which plane is substantially perpendicular to said main axis.

In an exemplary embodiment of the invention, said base section is curved and lies on either side of said main axis.

In an exemplary embodiment of the invention, said base section contacts said rail section at least three points, an axially middle one of said points being on an opposite side of said main axis than the other two of said points.

In an exemplary embodiment of the invention, said rail leg defines an aperture adapted to receive a suture.

There is also provided in accordance with an exemplary embodiment of the invention, a method of deploying a clip, comprising:

engaging vascular tissue using a hooked section of a rail leg;

sliding a hooked sliding leg along said rail leg until it engages said tissue;

locking said sliding leg to said rail leg; and

tearing a section of said rail leg off adjacent a locked location of said sliding leg.

### BRIEF DESCRIPTION OF THE DRAWINGS

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1 is a plan view of a rail leg of a sliding clip in accordance with an exemplary embodiment of the invention;

FIG. 2 is a plan view of a sliding leg of a sliding clip in accordance with an exemplary embodiment of the invention;

FIG. 3 is a perspective view of the rail leg of FIG. 1, bent in accordance with an exemplary embodiment of the invention;

FIG. 4 is a perspective view of the sliding leg of FIG. 1, bent in accordance with an exemplary embodiment of the invention;

FIG. 5 is a perspective view of an assembled sliding clip, in accordance with an exemplary embodiment of the invention;

FIG. 6 is a perspective view of a locked sliding clip, in accordance with an exemplary embodiment of the invention;

FIG. 7 is a perspective view of a locked sliding clip having a sliding leg mounted in parallel to a rail leg, in accordance with an exemplary embodiment of the invention;

FIG. 8 is a perspective view of a locked sliding clip having a sliding leg mounted using a curved mount onto to a rail leg, in accordance with an exemplary embodiment of the invention;

FIG. 9 is a perspective back view of a locked sliding clip similar to that of FIG. 8, in which a sliding leg has a forked spike, in accordance with an exemplary embodiment of the invention;

FIG. 10 is a perspective view of a sliding clip in which a sliding leg slides along a rail leg, and not coupled thereto, in accordance with an exemplary embodiment of the invention;

FIG. 11 is a perspective view of a sliding clip in which a sliding leg slides in a slot formed in a rail leg, in accordance with an exemplary embodiment of the invention;

FIG. 12A is a perspective view of an alternative sliding clip in which a sliding leg slides in a slot formed in a rail leg, in accordance with an exemplary embodiment of the invention;

FIG. 12B is a plan view of a sliding leg section of the clip of FIG. 12A;

FIG. 13 is a side view of a delivery system with only one clip shown, in accordance with an exemplary embodiment of the invention; and

FIG. 14 shows a leg tearing mechanism, in accordance with an exemplary embodiment of the invention.

### DETAILED DESCRIPTION OF EXEMPLARY EMBODIMENTS

Sliding Clip

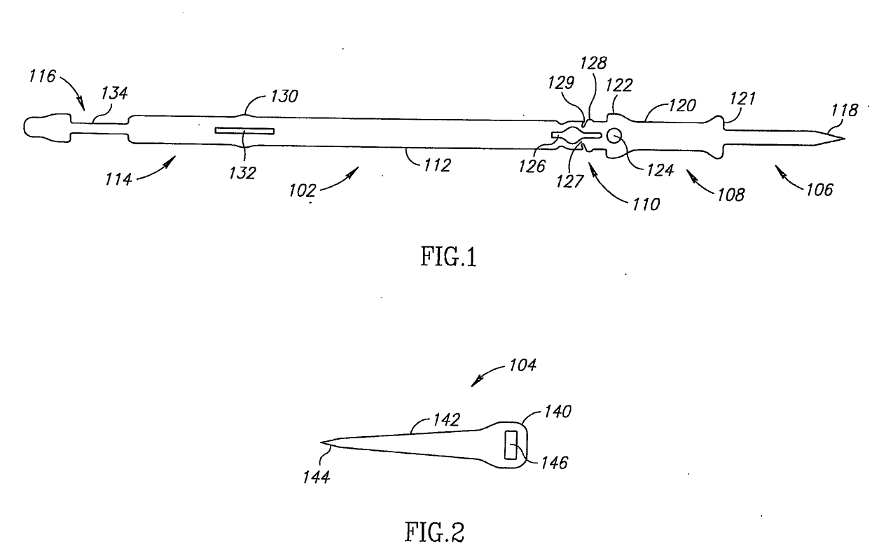

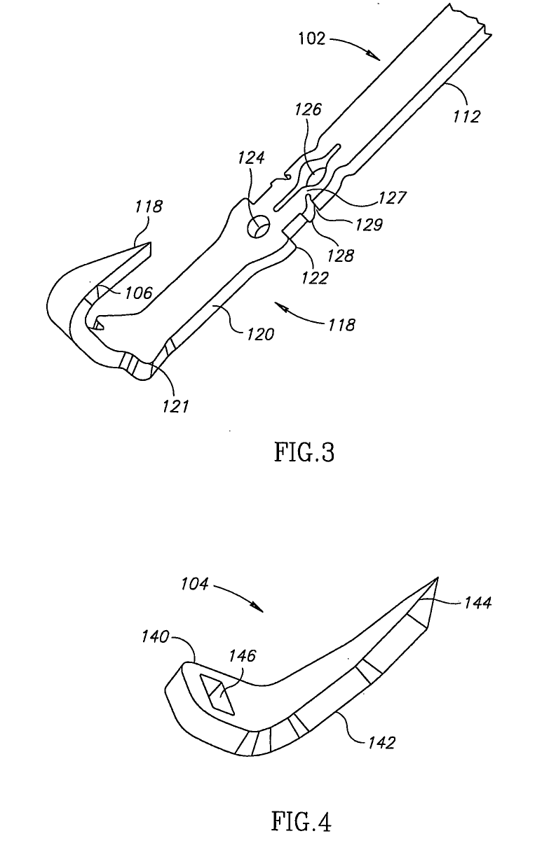

FIGS. 1-6 show a sliding clip in plan view, in parts and assembled, in accordance with an exemplary embodiment of the invention.

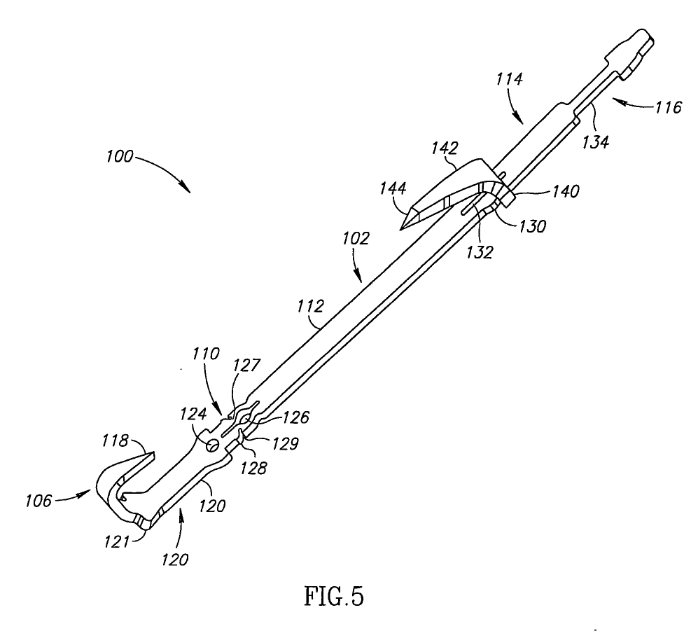

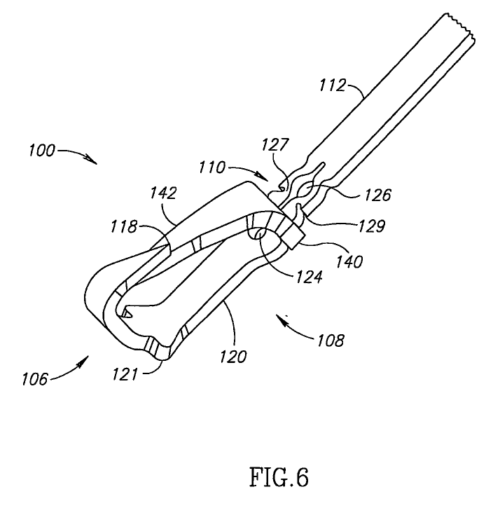

Referring first to FIGS. 5 and 6, a clip 100 is shown comprising a rail leg 102 and a sliding leg 104, which slides along rail leg 102. In FIG. 5, clip 100 is open and in FIG. 6, clip 100 has been slid shut, so that it is locked in position.

FIG. 1 shows a plan view of rail leg 102. As described below, in this embodiment, rail leg 102 comprises a forward section or tip 106, which is optionally bent into a hook as shown in FIGS. 5 and 6, a body section 108, whose length optionally corresponds to an anastomosis thickness, a locking position area 110 for sliding leg 104, a shaft 112, an unlock position area 114 and a retracting end 116. Each of these areas and some variations will now be described.

Tip 106 is optionally formed to have a single sharp tip 118, adapted to penetrate blood vessel tissue. Alternatively, other tip designs can be used, for example, a tip with a tissue stop to prevent over penetration. In another example, more than one tip 106 are provided, or a forking is provided in tip 106 near its base, such that each such tip is substantially free to penetrate tissue. Alternatively, tip 118 is adapted to not penetrate tissue, for example to lie flat against an inside wall of a blood vessel.

In an exemplary embodiment of the invention, tip 106 is bent so that sharp tip 118 is curved back and distanced a short distance from the rest of tip 106 and leg 102, for example to prevent tearing by piercing too close to an aperture lip. The length of the curved back portion may be selected, for example, to ensure transfixing of one or more layers of flesh or to prevent transfixing all the way through a blood vessel wall. As will be described below, sliding leg 104 may also include a tip. Optionally the tips are configured to meet inside the tissue. Alternatively, the tips are configured to slide past one another. Alternatively, the tips are configured to not reach one another.

Optionally, a tissue stop 121 is provided on rail leg 102, to prevent over penetration in tissue and/or later migration or rotation of the locked clip. Alternatively or additionally, tissue stop 121 is used to position an end of a graft vessel which is transfixed by tip 118.

Body 108 is shown as being a solid section 120. Alternatively, an open structure may be used. Optionally, section 120 is elongatabale in an elastic manner, which allows the clip to conform to various tissue thicknesses (e.g., by elongation) and/or to prevent over tightening of the tissue connection. For example, section 120 may be cut in the form of a folded ribbon or a cell structure. An aperture 124 is optionally provided through body 120. This aperture may be used, for example, to allow suturing by hand over an existing clip. Alternatively or additionally, the aperture is used for attaching multiple clips using a suture, which prevents them from drifting too far apart and/or for safety, to prevent a clip from falling into a blood stream. Optionally, the suture is removed after the anastomosis is complete and/or is bio-absorbable. Alternatively, a permanent suture is used. Optionally, the suture is elastic, to allow for some relative movement.

Optionally a tissue stop (not shown), further from tip 118 than stop 121, is provided in body 120 to position a graft vessel wall in a desired position.

Locking position area 110 is where sliding leg 104 rests when locked. Optionally, a plurality of axially displaced such positions are provided. A sliding stop 122 is optionally provided to prevent further sliding of leg 104. Alternatively the presence of tissue between the sharp tips prevents such sliding. One or more optional locking tabs 128 resist reverse sliding of leg 104 after it reaches area 110. The distance between stop 122 and tab 128 may be designed to match a thickness of sliding leg 104, so that sliding leg 104 is locked in a fixed position. Alternatively, some amount of freedom of motion is provided by increasing the distance. Optionally, the length of area 110 takes into account the presence of a wall of a graft vessel between stop 122 and sliding leg 104, when deployed.

In an exemplary embodiment of the invention, a slot 126 is provided between tabs 128, so that when sliding leg 104 is first advanced over rail leg 102, it pushes the tabs inwards towards the slot and thus fits by. Reverse motion is prevented, for example, either by lack of sufficient force or by a shape of tabs 128 which is inclined towards end 116 and flat towards tip 106.

In an exemplary embodiment of the invention, slot 126 is widened adjacent tabs 128 so that a thinner area 127 is provided in area 110. In an exemplary embodiment of the invention, this thinner area is torn when end 116 of leg 102 is pulled away from stop 122. Optionally, one or more tear-assisting slots or weakenings 129 are provided after tabs 128.

In an exemplary embodiment of the invention, weakened areas 127 are configured to assist in locking using tabs 128. For example, during tearing, weakened sections 127 elongate, along with material connected to them and forming an inner side of tabs 128. The outer sides of tabs 128 do not elongate, or at least do not elongate as much, causing tabs 128 to bend outwards.

Shaft 112 is optionally uniform, to prevent inadvertent locking of leg 104 on leg 102 and/or distortion thereof. Optionally, however, an intermediate semi-locked position for sliding leg 104 is provided near area 110, to allow the clip to be in a semi locked position, during an anastomosis process. Optionally, shaft 112 narrows towards area 110.

Prior to closing of clip 100, sliding leg 104 optionally rests at unlock position area 114. In an exemplary embodiment of the invention, inadvertent motion of sliding leg 104 is prevented by providing one or more tabs 130 with a corresponding slot 132. Tab 130, as shown, is designed to allow both forward and reverse motion of sliding leg 104 over rail leg 102.

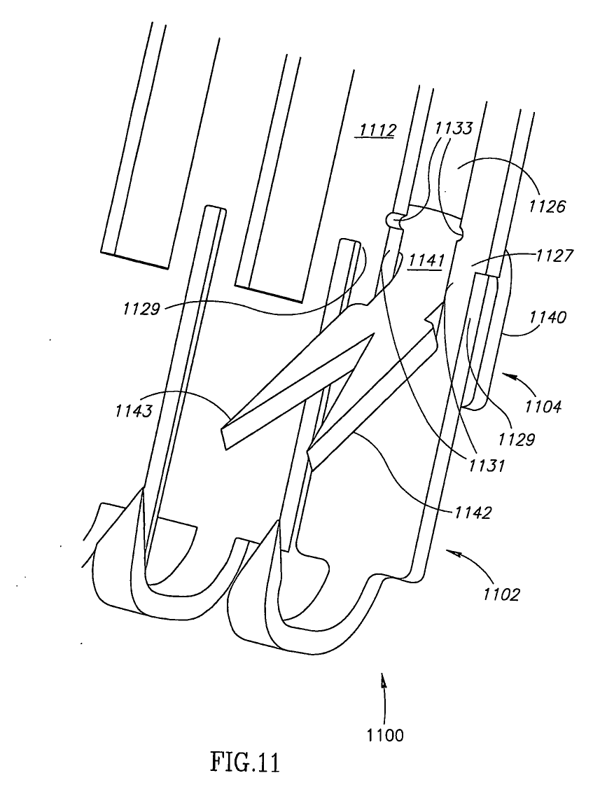

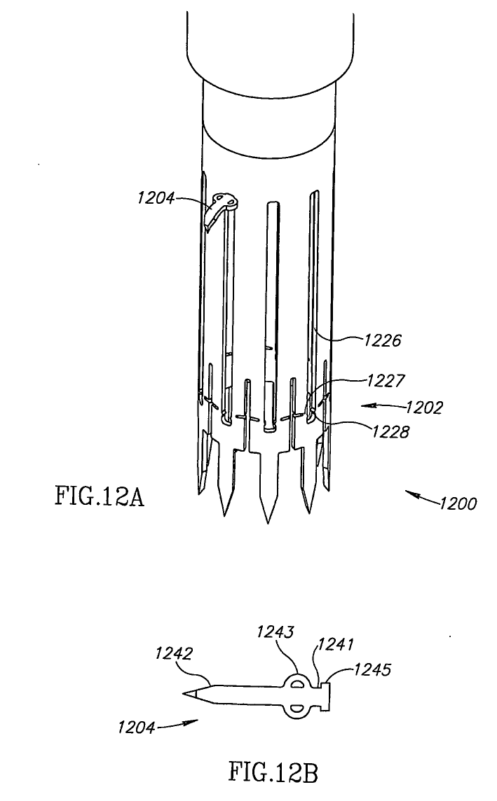

In an exemplary embodiment of the invention, rail leg 102 is retracted by a retraction mechanism. Optionally, rail leg 102 is engaged at a notch 134 defined therein. Alternatively, an aperture may be used for such engaging. Alternatively, for example, if the sliding leg is as shown in FIG. 11 or FIG. 12, rail leg 102 may be contiguous with a pulling mechanism.

FIG. 2 is a plan view of a sliding leg 104 in accordance with an exemplary embodiment of the invention. An eye section 140 includes an aperture 146 adapted to pass shaft 112 therethrough, so that the plane of 140 is perpendicular to the plane of shaft 112. In an exemplary embodiment of the invention, the fit of the shaft in the aperture has a close tolerance. Alternatively a loose fit is utilized.

A body section 142 of sliding leg 104 comprises a tip 144 which is optionally bent to penetrate a blood vessel wall, as shown in FIG. 6. While not shown, a tissue stop is optionally provided on body 142 to set the maximum penetration depth of tip 144. Optionally, tip 144 is forked, or more than one tip or body sections may be provided, to control penetration. Body 142 can have other shapes, for example, be a spiral or include a spring section which compresses in a plane of body 142. In an exemplary embodiment of the invention, sliding leg 104 is bent as shown in FIG. 4, to lie substantially parallel to shaft 112 and be distanced at a desired distance, various angles, for example, between 90 degrees and 20 degrees may be provided. Exemplary offsets (of tip 144 from shaft 112) are between 0.5 and 3 mm. For oblique connections between a vessel and a graft, for example, different ones of the clips in a set used for a single connection, may have different angles and/or offsets.

Variations

In the embodiment shown in FIGS. 1-6, eye section 140 is substantially perpendicular to shaft 112. However, an angle may be desired, for example, an angle of between 80 and 20 degrees or an angle between 100 and 150 degrees. This can be provided, for example, by providing an aperture having an axis that is not perpendicular to the surface of the eye section.

Leg 104 is shown to be adapted to penetrate a target blood vessel. In an exemplary embodiment of the invention, this adaptation is provided by forming or polishing (e.g., by electro-polish) tip 144 (and/or tip 118), so that it merely penetrates vascular tissue and does not tear it or cut it. In an alternative embodiment of the invention leg 104 is flat, for example as shown in PCT publication WO 03/026475, the disclosure of which is incorporated herein by reference. Alternatively, a bent body 142 is provided, but tip 144 is flattened so that it does not significantly penetrate tissue. Optionally body 142 is made elastic enough to assist in conforming to variations in thickness of a blood vessel wall.

Optionally, for example as shown in WO 03/026475, sliding leg 104 includes a tab that locks to an aperture in rail leg 102, for example, aperture 124.

In the embodiments of FIGS. 1-6, tabs are described as extending to the sides. Alternatively or additionally, such tabs comprise thickenings of the material, for example, if leg 102 is cut from a tube or a flat sheet.

Parallel Contact Clip

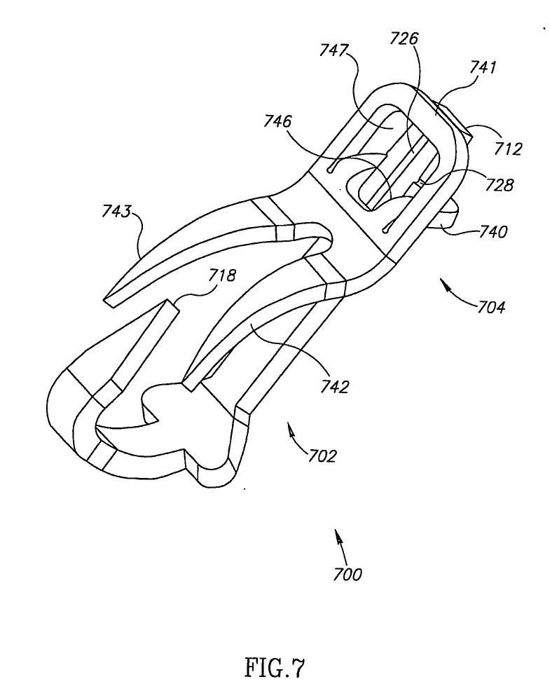

FIG. 7 is a perspective view of a locked sliding clip 700 having a sliding leg 704 mounted in parallel to a rail leg 702, in accordance with an exemplary embodiment of the invention. Sliding leg 704 has a base 741, enclosing an aperture 747. As shown, base 741 lies on a same side of rail leg 702, as a pair of spikes 742 and 743, forming a fork. Within aperture 747 a ring 740 is provided, with an aperture 746. Ring 140 and aperture 746 engage rail 702 as described above with reference to elements 140 and 146 in clip 100.

One potential advantage of this design, in some implementations thereof, is that sliding leg 704 engages rail leg 702 in a stable manner, at least with respect to rotation in a plane including rail leg 702 and spikes 742 and 743. This stability is provided by having two resting points on one side of leg 702 and one on the other side, between the other two. It should be noted that the clips shown are very small, so that an aperture such as aperture 146 provides only a limited amount of resistance to rotation, especially if a relatively loose fit is provided. Optionally, rotational stability in a plane along the direction of sliding is provided by having two spaced apart rings 740.

Another potential advantage of this design, in some embodiments of the invention, is that a lower tolerance may be used for manufacturing. For example, ring 740 and 741 may be elastically pre-disposed towards each other, so aperture 746 need be exact only in a dimension where it locks to leg 702 (e.g., a width dimension relative to locking tabs 728, described below).

In an exemplary embodiment of the invention, sliding leg 704 is mounted on a far side (not shown) of a body 712 of rail leg 704, which far side may have a same design as for clip 100. A slot 726 lies between two tabs 728, so that ring 740 can slide over tabs 728, by compressing the tabs towards slot 726. Optionally, a forward tab (not shown) is provided to prevent over advance. Alternatively, no such prevention is provided. Alternatively, slot 726 is defined along body 712 and body 712 has a width greater than the opening of aperture 740. Ring 740 slides over body 712 only by compressing body 712 inwards towards slot 726.

While a sliding ring 740 is shown, in an alternative embodiment of the invention, an inner tab, is provided instead, which tab slides along slot 726, for example as described below for other embodiments. Tearing may be provided using slots as for clip 100, above, for example.

Clip 700 also shows a variation on the clip structure in that sliding leg 704 includes a forked tip having two extensions, 742 and 743, which may, for example, fit on either side of a tip 718 of leg 702. Alternatively or additionally, leg 702 has two or a higher number of tips.

In an exemplary embodiment of the invention, for this or other embodiments, the provision of multiple extensions (e.g., forked tip pair 743 and 742) is used to assist in prevent dissection of the blood vessels being attached. However, this is not a required feature of the invention.

Curved Engagement Clip

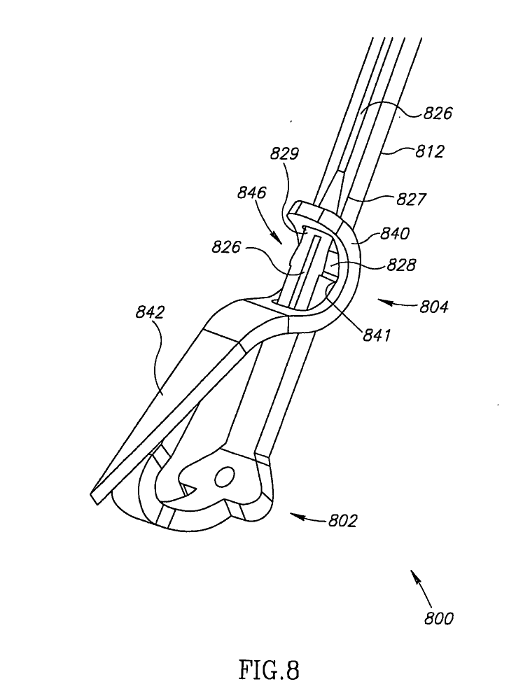

FIG. 8 is a perspective view of a locked sliding clip 800 having a sliding leg 802 mounted using a curved mount 840 onto to a rail leg 804, in accordance with an exemplary embodiment of the invention. The reference numbers generally correspond to those of FIG. 7.

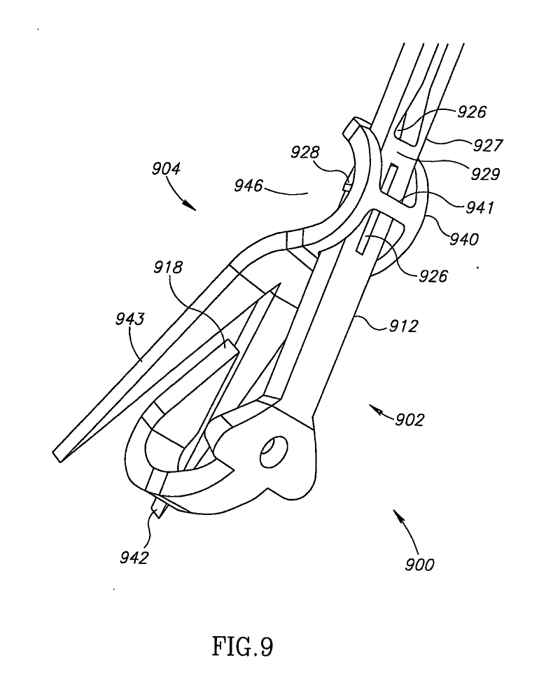

Like clip 700, also clip 800 has a three point locking mechanism A sliding leg section 804 includes a base ring 840 which is bent so that a front and a back end thereof are on one side of a rail leg 802 and a middle is on the other side. Optionally, a back bar 841 is provided to prevent ring 840 from slipping off. In an exemplary embodiment of the invention, a single slot 826 is provided. Optionally, a bar 829 separates the slot into to separate slots, for example, to prevent blood leakage along the slot or to control the elasticity of the slot. Optionally, bar 829 serves as a stop against which tearing forces may be applied. FIG. 9 is a perspective back view of a similar clip 900 having a forked sliding leg, in which a back bar 941 is clearly visible.

A potential advantage of this design is that there are fewer sharp edges and/or bends, as compared to that of clip 700.

Tab Locking Clip

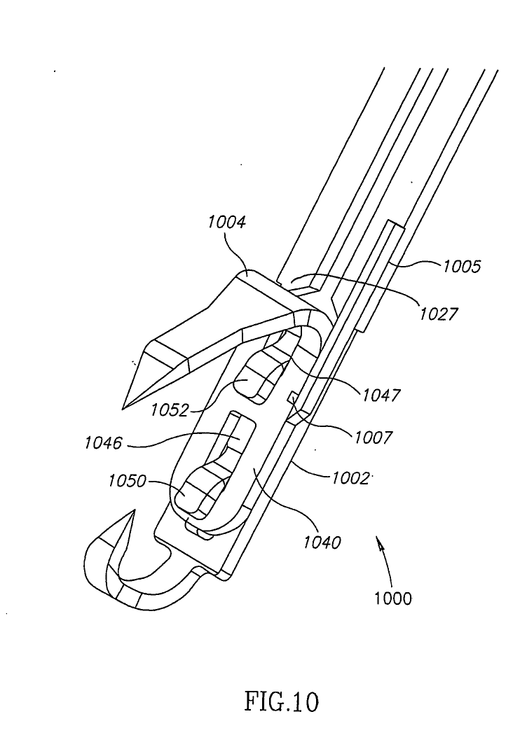

FIG. 10 is a perspective view of a sliding clip 1000 in which a sliding leg 1004 slides along a rail leg 1002, and not coupled thereto, in accordance with an exemplary embodiment of the invention. It should be noted that in some methods of delivery, sliding leg 1004 (or its corresponding legs in other embodiments) does not move relative to a base holding the leg. Instead leg 1002 is retracted. In an exemplary embodiment of the invention, in such a delivery system, sliding leg 1004 is held in place by a fork having a tine on either side of the leg. An exemplary tine 1005 is shown, and includes an optional step 1007 to prevent leg 1004 from moving away. As shown, leg 1004 includes two apertures 1046 and 1047 in a base section 1040 thereof. A pair of elastic tabs 1050 and 1052 are formed in rail leg 1002. When base 1040 slides over tabs 1050 and 1052, they elastically compress inwards. When base 1040 passes tabs 1050 and 1052 spring back. The presence of tissue in the clip generally pushes leg 1004 back, where it is locked by tabs 1050 and 1052. In an alternative embodiment, one or both tabs is formed on sliding leg 1004 rather than on rail leg 1002. Alternatively or additionally, only one tab or more than two tabs are used. Optionally, each tab can serve to define a locking station for the clip, for example, allowing multiple locking positions to be achieved by selectively advancing leg 1004 more along leg 1002.

Leg-In-Slot Clip

FIG. 11 is a perspective view of a sliding clip 1100 in which a sliding leg 1104 slides in a slot 1126 formed in a rail leg 1102, in accordance with an exemplary embodiment of the invention. Instead of a ring section of the sliding leg fitting over the rail leg, a tab section 1141 of sliding leg 1104 fits in slot 1126, with a base section 1140 on an opposite side of leg 1102 than a pair of forked penetrating tips 1142 and 1143. This has a potential advantage of having a smaller width profile and/or fewer variations in width of the deployed clip. Optionally, the sliding leg both engages the rail leg from outside and from a slot in the rail leg.

In an exemplary embodiment of the invention, rail legs 1102 are mounted on extensions 1112. Unlike previous embodiments, where the extensions were each shown for a single rail leg, in FIG. 11, each extension 1112 bridges two rail legs 1102. The coupling between extensions 1112 and legs 1102 optionally forms a weakening 1127, which is torn. In an exemplary embodiment of the invention, weakening 1127 is designed so that an inner side 1131 tears first and then an outer side 1129 tears. Thus, inner side 1131 is urged into slot 1126, by outer side 1129 having been stretched more, and locks tab 1141. For example a cut-out section 1133 may serve to control the tearing order.

Alternative Leg-In-Slot Clip

FIG. 12A is a perspective view of an alternative sliding clip 1200 in which a sliding leg 1204 slides in a slot 1226 formed in a rail leg 1204, in accordance with an exemplary embodiment of the invention. FIG. 12B is a plan view of sliding leg 1204, showing a tip 1242 thereof, which is curved in FIG. 12A. In this embodiment, sliding leg 1204 fits into slot 1126 in a perpendicular manner, with a tab 1241 fitting in slot 1126 and a stop 1245 and a second stop 1243 fitting on either side of rail leg 1204. Optionally, one or both of stops 1245 and 1243 are elastically urged towards the other stop, to better engage rail leg 1202 and/or stabilize sliding leg 1204. Optionally, one or more inner tabs 1228 are formed in slot 1226 to lock tab 1241 and thus leg 1204 in place. A weakening 1227 is shown.

In an exemplary embodiment of the invention, tearing of weakening 1227 is provided by pulling back on the rail legs relative to the sliding legs. A similar mechanism may be used in the other embodiments as well. This may impose high strains on the sliding leg. In an exemplary embodiment of the invention, the sliding leg is backed by a ring (not shown) which prevents the sliding leg from distorting. Optionally, an extension of such a ring is provided in slot 1226, to hold the sliding leg in place and prevent its distortion. In this and other embodiments, the structure of the rail leg is optionally made strong enough to resist distortion except for weakenings 1227. For example, in FIG. 1, stop 122 is made robust. For example, the tearing force may be 0.5, 1, 5, 10 or 25 Kg, or intermediate or greater values. For example, a force of 25 Kg is used to tear 10 legs, so the force on a single leg is 2.5 Kg. In other examples, 6, 8 or 12 legs are torn. The tearing force per leg can be, for example, between 0.5 and 3 Kg, depending on the leg design.

In an exemplary embodiment of the invention, a staggered delivery system is used which tears fewer than all the legs at one time, thus reducing the total force required, for example, reducing the force which is applied by a human, a spring, a lever or an engine to less than 3 Kg. This is described below. Alternatively, clips 100 themselves are designed to tear in a serial manner, rather than all together. For example, by varying the distance between tip 118 (or stop 121) and stop 122, or by varying the thickness of leg 104, even if all the legs are pulled back together, the force will be applied first to those legs whose stop 122 is closer to pulling section 116.

In one embodiment of the invention, the clips are arranged so that opposing clips will tear together. Alternatively or additionally, the clips are arranged to tear in a circle. Optionally, the clips are arranged to tear in accordance with an oblique connection or to tear at ends of an incision first or last.

In an alternative staggered tearing delivery system, lengths of shafts 112 are varied. Alternatively or additionally, a ring holding areas 116 is modified so that it engages each area at a different point. Alternatively or additionally, a ring pressing down on legs 104 is varied to have a different relative displacement for different clips.

Delivery System

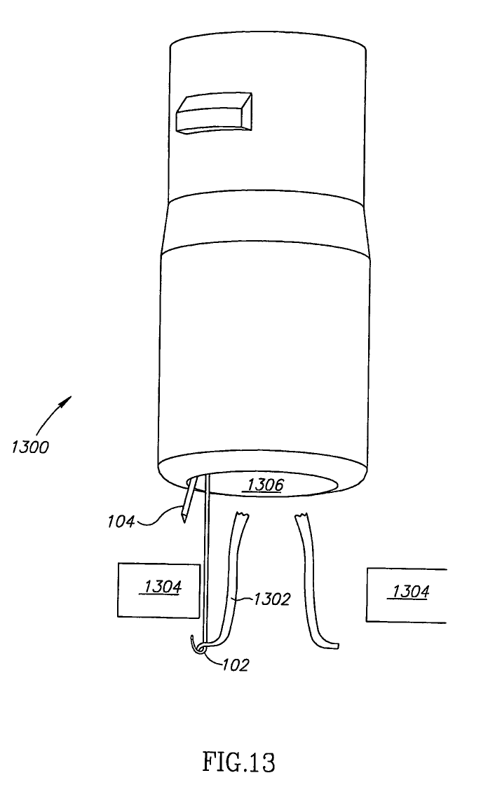

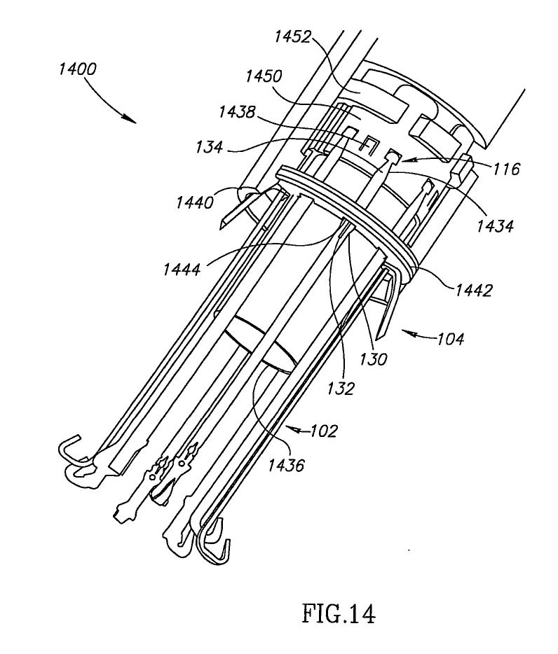

FIG. 13 is a side view of a delivery system 1300 with only one clip shown, in accordance with an exemplary embodiment of the invention. Delivery system 1300 has a front aperture 1306 through which a plurality of clips can extend. For clarity, only a single clip is shown and only a section of a graft 1302 is shown everted on a rail leg 102. A sliding leg 104 is shown distanced from rail leg 102. In use, rail leg 102 is retracted, engaging a target vessel 1304. Then, sliding leg 104 is advanced, to enter the wall of vessel 1304 and then lock to rail 102. Then leg 102 is torn. In a simplest system, this is all achieved by retracting leg 102, effectively sliding leg 104 on it. Thus, many of the systems described in earlier application by the instant applicant and by other applicants can be used.

FIG. 14 shows the inside of an exemplary delivery system 1400 for a clip 100. Two sliding legs 104 are shown in temporary locking areas defined by tabs 130 and slot 132. A back ring 1442 is provided to hold legs 104 in place and to serve as a backing while applying a the tearing force thereto. An over tube 1440 is optionally provided to help maintain a proper orientation of the legs, for example while penetrating tissue. As shown, legs 102 fit between an inner tube 1436 and a slot 1444 formed in ring 1442. In the embodiment shown, legs 102 are held by a slotted plate 1450. A plurality of slots 1434 are shaped to each engage a notch 134 of a holding area 116. A covering tube, not shown, prevents holding areas 116 from falling out of the slots. Plate 1450 is retracted by a mechanism ending in couplers 1452. In one embodiment, ring 1442 may be advanced past tube 1436. In this case, when retracting the systems, one or more tabs 1438 engages and holds onto tube 1436 so that it moves with plate 1450. Plate 1450 optionally comprises two plates, one on either side of the delivery system, to support splitting.

In particular, the following documents, the disclosures of which are incorporated herein by reference describe connectors, delivery systems and/or other tools and methods which are useful in conjunction with embodiments of the prevent invention:

PCT/IL02/00790, filed on Sep. 25, 2002, now published as WO 03/026475;

U.S. Ser. No. 60/492,998 filed on Aug. 7, 2003.

PCT/IL02/00215, filed on Mar. 18, 2002, now published as WO 02/074188;

PCT/IL01/01019, filed on Nov. 4, 2001, now published as WO 02/47532;

PCT/IL01/00903, filed on Sep. 25, 2001 now published as WO 02/30172;

PCT/IL01/00600, filed on Jun. 28, 2001, now published as WO 02/47561;

PCT/IL01/00267, filed on Mar. 20, 2001, now published as WO 01/70091;

PCT/IL01/00266, filed on Mar. 20, 2001, now published as WO 01/70090;

PCT/IL01/00074, filed on Jan. 25, 2001, now published as WO 01/70119;

PCT/IL01/00069, filed on Jan. 24, 2001, now published as WO 01/70118;

PCT/IL00/00611, filed on Sep. 28, 2000, now published as WO 01/41624;

PCT/IL00/00609, filed on Sep. 28, 2000, now published as WO 01/41623,

PCT/IB00/00310, filed on Mar. 20, 2000, now published as WO 00/56228;

PCT/IB00/00302, filed on Mar. 20, 2000, now published as WO 00/56227;

PCT/IL99/00674, filed on Dec. 9, 1999, now published as WO 00/56223;

PCT/IL99/00670, filed on Dec. 8, 1999, now published as WO 00/56226;

PCT/IL99/00285, filed on May 30, 1999, now published as WO 99/62408; and

PCT/IL99/00284, filed on May 30, 1999, now published as WO 99/62415. The disclosure of all of these applications, which designate the US and were filed in English, are incorporated herein by reference.

In addition, a PCT application filed on same date with the present application, by applicant “By-Pass Inc.”, and describing anastomotic connectors is “Anastomotic Connectors”, attorney docket number 088/03736, the disclosure of which is incorporated herein by reference. A PCT application filed on same date with the present application, by applicant “By-Pass Inc.”, and describing leg arranging systems is “Anastomotic Leg Arrangement”, attorney docket number 088/03504, the disclosure of which is incorporated herein by reference.

A provisional application filed on even date with the instant application, by applicants Loshakove, et. al and having attorney docket number 088/03695 and title “Bypass Punch Anastomosis Delivery System” is also incorporated herein by reference and describes an exemplary delivery system.

A clip may be manufactured of various materials, including for example, metals (e.g., stainless steel, NiTi alloys and titanium), plastics and bio-absorbable materials. Optionally, the clip is formed of a material that exhibits elastic, super elastic and/or shape memory properties.

Some of these applications describe anastomosis delivery systems and hole making apparatus and/or other device useful in cooperation with the present invention. Some of these applications describe delivery systems in which separate steps are provided for retracting and tearing, and even, in some embodiments, for advancement of legs 104.

The above described sliding clips may be varied in many ways. For example, the rail legs and/or the sliding legs may be interconnected before or after the anastomosis, for example, using a flexible element, such as a suture, or a rigid element, such as a metal bar.

In an exemplary embodiment of the invention, the tips of the sliding leg and the rail leg are sharpened to minimize trauma to the blood vessels, during attaching, and especially to reduce tearing and/or dissection. For example, the tips may be formed to be needle like, so that they have no edges that can tear nearby tissue, except when inserted, tip first.

While the above clips have been described in general for any type of blood vessel, it should be appreciated that particular modifications may be desired for certain vessel types. For example, the aorta is thicker, while a coronary vessel is thinner, thus suggesting different amounts of space in the clip. For example, an aorta may be 3 mm thick, while a coronary vessel may be less than 1 mm thick.

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. The exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A sliding surgical clip adapted to connect blood vessel tissue, comprising:
an elongate rail leg having a main axis and terminating with a first tissue penetrating tip; and
a sliding leg terminating with a second tip and configured to slide along said rail leg towards said first tissue penetrating tip, such that said two tips face each other and engage vascular tissue between them.

2. A clip according to claim 1, wherein said second tip comprises a tissue penetrating tip.

3. A clip according to claim 1, comprising a tissue stop on said sliding leg, which stop prevents over penetration of said leg into vascular tissue.

4. A clip according to claim 2, wherein said second tip is forked.

5. A clip according to claim 2, wherein said second tip has a section between 0.5 mm and 5 mm long adapted to enter a blood vessel.

6. A clip according to claim 2, wherein said second tip has a section between 1 mm and 4 mm long adapted to enter a blood vessel.

7. A clip according to claim 2, wherein said second tip has a section between 0.5 mm and 3 mm long adapted to enter a blood vessel.

8. A clip according to claim 1, wherein said first tip has a section between 0.5 mm and 4 mm long adapted to enter a blood vessel.

9. A clip according to claim 1, wherein said first tip is forked.

10. A clip according to claim 1, wherein said first leg and said second leg are narrower than 1 mm, in a widest dimension thereof, for at least 3 mm adjacent their tips.

11. A clip according to claim 1, wherein said first leg and said second leg are narrower than 0.7 mm, in a widest dimension thereof, for at least 2 mm adjacent their tips.

12. A clip according to claim 1, wherein said first leg and said second leg are narrower than 1 mm, in a widest dimension thereof, for at least 20 mm adjacent their tips.

13. A clip according to claims 1, wherein said clip is adapted for use with a blood vessel having a diameter of between 2 mm and 40 mm.

14. A clip according to claim 1, wherein said clip is provided as a set of connectors arranged in a generally circular array and adapted for use for attaching a blood vessel having a diameter of between 1 mm and 10 mm to a second blood vessel.

15. A clip according to claim 14, wherein said set is sutured together at their rail sections.

16. A clip according to claim 1, wherein said second tip is adapted to penetrate vascular tissue without tearing the tissue.

17. A clip according to claim 1, wherein said elongate rail leg comprises a lock which prevents reverse sliding of said sliding leg.

18. A clip according to claim 17, wherein said elongate rail leg comprises multiple lock locations which prevents reverse sliding of said sliding leg.

19. A clip according to claim 17, wherein said lock comprises a transaxial extension of said elongate rail which is configured to elastically move out of the way when said sliding leg slides towards said first tip.

20. A clip according to claim 19, wherein said rail includes a slot adjacent said extension, to support said elastic motion.

21. A clip according to claim 19, wherein said transaxial extension is robust enough to withstand a force of at least 1 Kg on said extension.

22. A clip according to claim 1, wherein said elongate rail leg comprises a lock which prevents forward sliding of said sliding leg after being locked.

23. A clip according to claim 1, comprising a tissue stop on said rail leg to prevent over-penetration of said leg into vascular tissue.

24. A clip according to claim 1, wherein said rail leg defines a weakened location adapted to be selectively torn when sufficient force is applied to opposite sides of said weakened location.

25. A clip according to claim 1, wherein said rail leg defines a temporary locking location distanced from said tip, configured to hold said sliding leg prior to said sliding.

26. A clip according to claim 1, wherein said sliding leg engages said rail leg from its outside.

27. A clip according to claim 1, wherein said rail leg defines a slot along its length and wherein said sliding leg engages said rail leg from said slot.

28. A clip according to claim 27, wherein said rail leg shares an elongate section with an adjacent rail leg, on an opposite side of a tearing location from said tip.

29. A clip according to claim 1, wherein said sliding leg includes a base section coupled to said rail leg.

30. A clip according to claim 29, wherein said base section defines an aperture, which aperture fits around said rail leg.

31. A clip according to claim 30, wherein said fit is snug.

32. A clip according to claim 29, wherein said base section lies in a plane, which plane lies parallel to said main axis.

33. A clip according to claim 29, wherein said base section lies in a plane, which plane is substantially perpendicular to said main axis.

34. A clip according to claim 29, wherein said base section is curved and lies on either side of said main axis.

35. A clip according to claim 29, wherein said base section contacts said rail section at least three points, an axially middle one of said points being on an opposite side of said main axis than the other two of said points.

36. A clip according to claim 1, wherein said rail leg defines an aperture adapted to receive a suture.

37. A method of deploying a clip, comprising:
engaging vascular tissue using a hooked section of a rail leg;
sliding a hooked sliding leg along said rail leg until it engages said tissue;
locking said sliding leg to said rail leg; and
tearing a section of said rail leg off adjacent a locked location of said sliding leg.
